(19) United States
(12) Patent Application Publication
(10) Pub. No.: US 2013/0073027 A1
(43) Pub. Date: Mar. 21, 2013

(54) STENT GRAFT WITH FLANGED CONTRALATERAL GATE FOR ENDOVASCULAR ANEURYSM REPAIR

(71) Applicant: Nikola Dobrilovic, Providence, RI (US)

(72) Inventor: Nikola Dobrilovic, Providence, RI (US)

(21) Appl. No.: 13/611,945

(22) Filed: Sep. 12, 2012

Related U.S. Application Data
(60) Provisional application No. 61/535,729, filed on Sep. 16, 2011.

(51) Int. Cl. A61F 2/06

(52) U.S. Cl. 623/1.13

(57) ABSTRACT

A stent graft is disclosed. The stent graft includes a body portion having a sidewall and an opening at the top. The top of the sidewall is configured and arranged to engage a wall of an aorta above an aneurysmal site of a patient. An ipsilateral limb descends from the body portion. The ipsilateral limb is configured and arranged to descend into one of an iliac arteries. The stent graft further includes a contralateral gate that has a flared opening. The contralateral gate is configured and arranged to align with the other of the iliac arteries.

### CROSS-REFERENCE TO RELATED APPLICATION

This patent document claims priority to earlier filed U.S. Provisional Application Ser. No. 61/535,729, filed on Sep. 16, 2011, the entire contents of which are incorporated herein by reference.

### BACKGROUND

1. Technical Field

The present patent document relates generally to stents and more specifically to a stent graft with a flanged contralateral gate for Endovascular Aneurysm Repair (“EVAR”).

2. Background of the Related Art

During an EVAR procedure, access of the contralateral gate of a stent graft to place the contralateral limb in place can often be one of the more challenging and time consuming aspects of the procedure. This vital portion of the case is, in a best case scenario, unpredictable because the physician's view of the procedure and control of the catheter and wires is limited. Surgeons devote a significant amount of time and effort, both preoperatively and intraoperatively, planning the insertion, position, orientation, and approach to the contralateral gate to ensure rapid cannulation. Such planning involves determining the rotation and angle of the main body of the stent graft to ensure easier cannulation.

In some situations, failure to access the contralateral gate can result in prolonged procedure time which results in increased risk to patients because of increased anesthesia time, creation of additional access sites, and other well-known complications that result in increased risk to both patients and operating room or catheterization laboratory (“cath lab”) team. For instance, increased duration of radiation exposure due to increased operative time.

Furthermore, the increased operating time results in significant increases to the cost of the operation. Significant costs are incurred for each minute of cath lab or operating room time. Also, increased cost is incurred by the need for additional special tools, such as angled catheters and wires that are used for wire manipulation, that would not be required if access to the contralateral limb was obtained easily.

On rare occasions, when contralateral gate access is extremely difficult, case complexity can escalate and require, for example, that the radial artery be accessed to provide the ability for antegrade graft cannulation and subsequent snaring from below the graft on the contralateral limb side.

Such results, of course, are highly undesirable. Therefore, there is a need for a method of consistently ensuring accurate and successful access to the contralateral gate to prevent excess operating room time and risk to the patient. Also, there is a need for a method to reduce surgical and surgical effort in planning stent graft cannulation.

### SUMMARY

The present invention solves the problems of the prior art by providing a flared or flanged opening on the contralateral gate to enhance access thereto. The flared or flanged end functions as a funnel that helps guide the wire into the stent graft for proper positioning and deployment of the contralateral limb. The use of the flared or flanged opening can also decrease the chance of contralateral limb deployment outside of the body portion of the stent graft. Also, during emergency situations, such as aneurysm rupture, the flared and flanged end can speed insertion of the stent graft and repair of the rupture. Significant time can be saved when even seconds are critical.

### BRIEF DESCRIPTION OF THE DRAWINGS

These and other features, aspects, and advantages of the present invention will become better understood with reference to the following description, appended claims, and accompanying drawings where:

FIG. 1a shows a view of a first embodiment of a stent graft positioned in the aneurysmal area of the abdominal aorta with the contralateral limb not deployed;

FIG. 1b shows a view of a first embodiment of a stent graft positioned in the aneurysmal area of the abdominal aorta with the contralateral limb deployed;

FIG. 2a shows a view of a second embodiment of a stent graft positioned in the aneurysmal area of the abdominal aorta with the contralateral limb not deployed;

FIG. 2b shows a view of a second embodiment of a stent graft positioned in the aneurysmal area of the abdominal aorta with the contralateral limb deployed; and

FIG. 3 shows a view of a third embodiment of a stent graft with a medial flare at the contralateral gate.

### DESCRIPTION OF THE PREFERRED EMBODIMENT

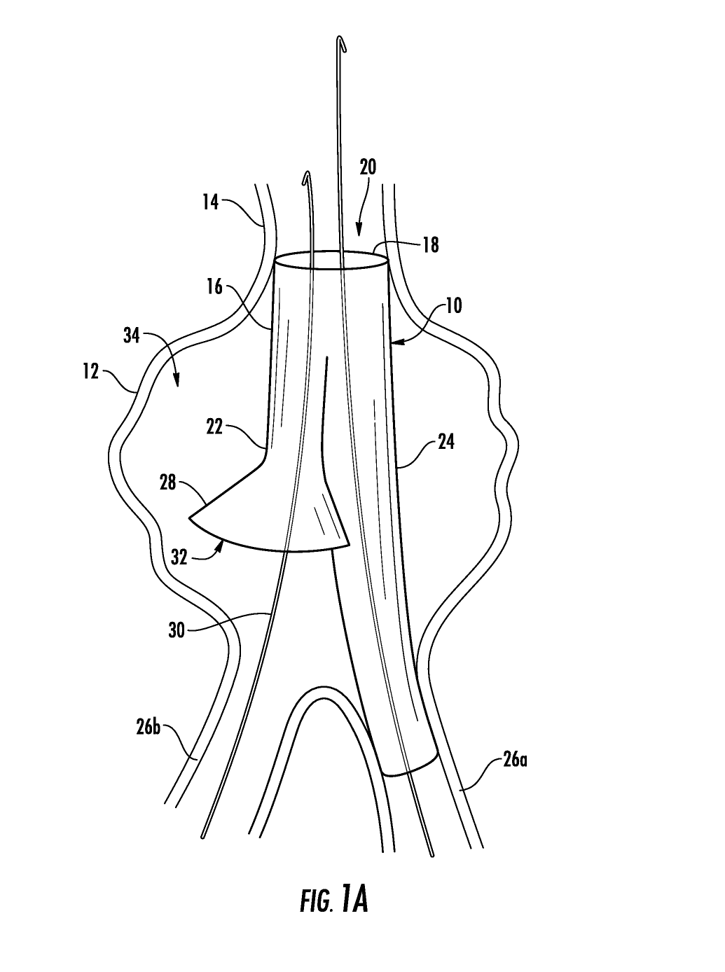

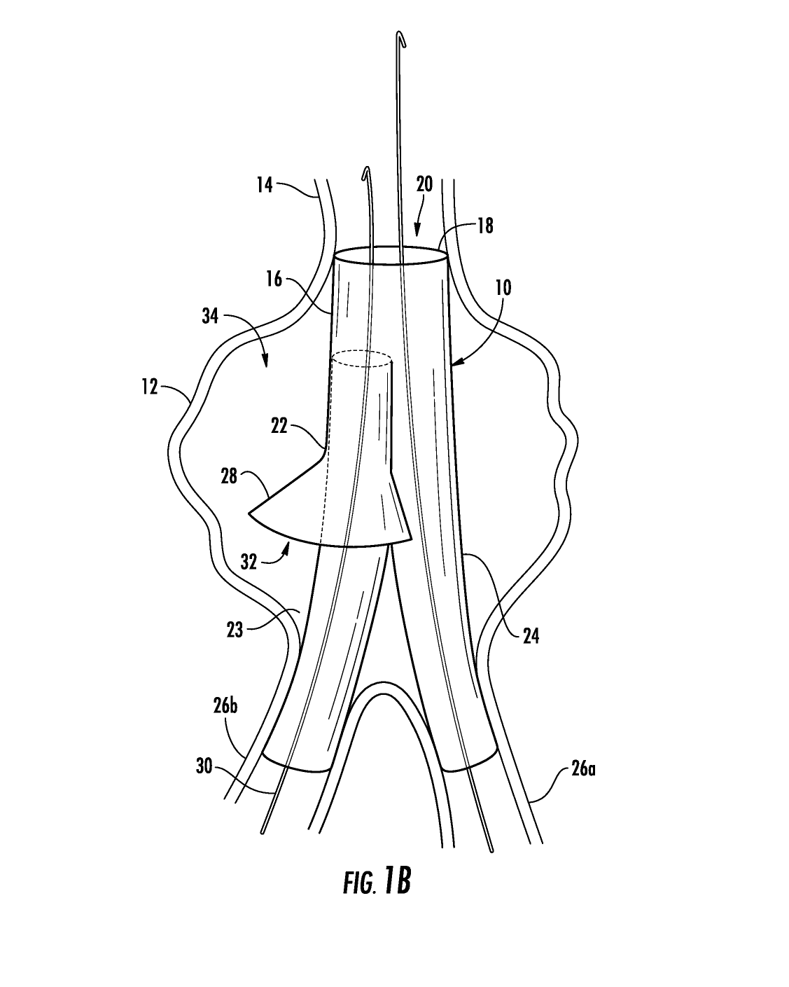

Referring now to FIGS. 1a and 1b, a first embodiment of the stent graft is shown generally at 10 in the aneurysmal site 12 of the abdominal aorta 14. The stent graft 10 includes a body portion 16 with a sidewall 18 and an opening 20 at the top. The top of the sidewall 18 engages the wall of the aorta 14 above the aneurysmal site 12. The stent graft 10 tapers to a contralateral gate 22 having an opening and an ipsilateral limb 24. The ipsilateral limb 24 is configured to descend into one of the iliac arteries 26a, 26b. The contralateral gate 22 is aligned with the other iliac artery 26b.

The stent graft 10 is shown deployed with the ipsilateral limb 24 descending into the left iliac artery 26a; however, the physician could deploy the stent graft 10 with the ipsilateral limb 24 descending down the right iliac artery 26b instead and the contralateral gate 22 aligned with the left iliac artery 26a.

The contralateral gate 22 includes an extra length of material 28 that is added or incorporated to facilitate wire 30 access to the stent graft 10. This additional material 28 provides considerable benefit with little or no added risk. The additional length of material 28 is flared outwardly and forms a flange or funnel shape 32 to guide the wire 30 into the contralateral gate 22.

By design, the flange 32 may be (though not necessarily) an extension of the same material from which the stent graft 10 is made. The flange 32 may include an additional support system, such as the wires from, or separate from, the support system of the primary stent graft 10. The open edge of the contralateral gate may have a radio-opaque marker to assist with radiographic imaging (as is commonly done).

The shape of the flange 32 may take a variety of actual forms. The flange 32 may also include the proximal portion of the ipsilateral limb 24, but may also be formed separately therefrom. Unlike the proximal and distal ends of stent graft 10, the “flare” or funnel's 32 primary role would not be to function in providing a seal with the vessel wall of the iliac artery 26b, or a contact point with other grafts, but rather to facilitate 30 wire access of the contralateral limb gate 22. Therefore, it is preferable that the flange 32 be located inside the aneurysm sac 12 where it can fully open. Once accessed, the flange 32 would essentially remain out of the way for the remainder of the EVAR procedure. The flange 32 would not significantly affect deployment/function of the contralateral limb 23.

Although the flange 32 could limit space within the aneurysm 12 which is required for access of the contralateral limb 23, this situation is unlikely because the two-part aortic stent graft is, by design, located in a relatively “high” position in the aneurysm sac 34. A shorter length of “flaring” with a wider angle of the flange should minimize or negate this risk.

Although the flange 32 has potential to dislodge debris from the aneurysm wall 12, this increased (theoretical) risk is likely trivial in actuality. In fact, overall risk of dislodging debris is likely reduced significantly as a result of easier contralateral limb wire 30 access. Easier access of the contralateral limb 23 should translate directly into much less wire 30 manipulation, which is, in fact, a known (and much more likely) cause of debris dislodgment with its known associated complications.

Furthermore, the size of the flare 32 can be reduced in order to allow full deployment in small aneurysms and tighter spaces. The flare 32 may also be made shallow to accommodate smaller aneurysm and tighter spaces.

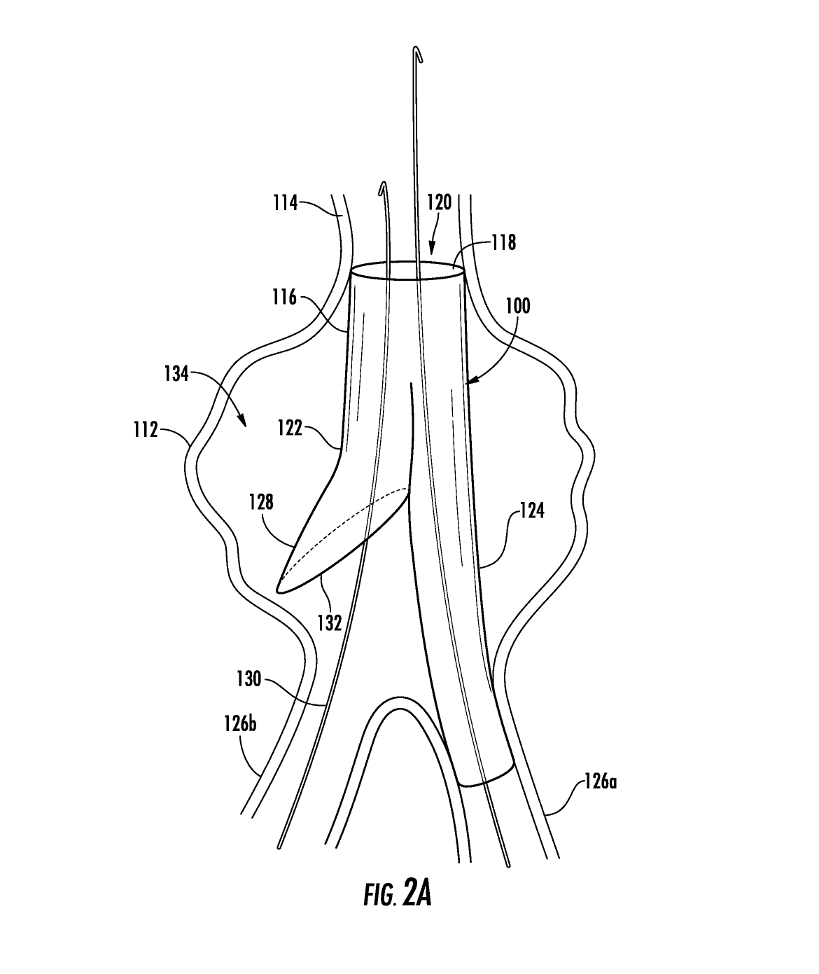

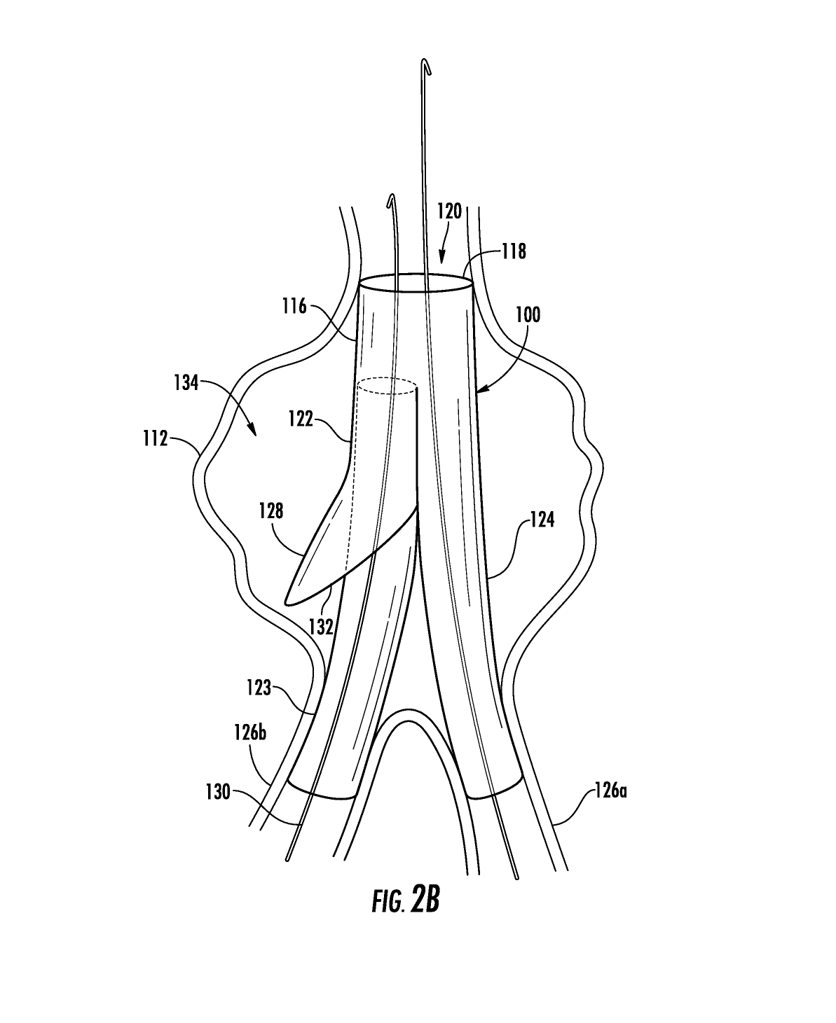

Referring now to FIGS. 2a and 2b, a second embodiment of the stent graft is shown generally at 100 in the aneurysmal site 112 of the abdominal aorta 114. The stent graft 100 includes a body portion 116 with a sidewall 118 and an opening 120 at the top. The top of the sidewall 118 engages the wall of the aorta 114 above the aneurysmal site 112. The stent graft 100 tapers to a contralateral gate 122 having an opening and an ipsilateral limb 124. The ipsilateral limb 124 is configured to descend into one of the iliac arteries 126a, 126b. The contralateral gate 122 is aligned with the other iliac artery 126b.

The stent graft 100 is shown deployed with the ipsilateral limb 124 descending into the left iliac artery 126a; however, the physician could deploy the stent graft 100 with the ipsilateral limb 124 descending down the right iliac artery 126b instead and the contralateral gate 122 aligned with the left iliac artery 126a.

The contralateral gate 122 includes an extra length of material 128 that is added or incorporated to facilitate wire 130 access to the stent graft 110 that includes a flanged or flared end 132. The flange or flared end 132 on the second embodiment 100 includes a beveled shape for the funnel that tapers towards the ipsilateral limb 124. For example, a large bevel on the medial (near junction of ipsilateral limb 124) or lateral side would allow a larger surface/target toward which the wire 130 could be aimed. This will allow improved wire 130 direction for contralateral gate 122 cannulation, yet still leave adequate opening above the origin of the contralateral iliac artery 126b. It is important to note that the flare 132 may be configured to flare in another direction, such as towards the ipsilateral limb 124 (i.e. medially as described below), laterally, anteriorly or posteriorly as required.

Furthermore, the size of the flare 132 can be reduced in order to allow full deployment in small aneurysms and tighter spaces. The flare 132 may also be made shallow to accommodate smaller aneurysm and tighter spaces.

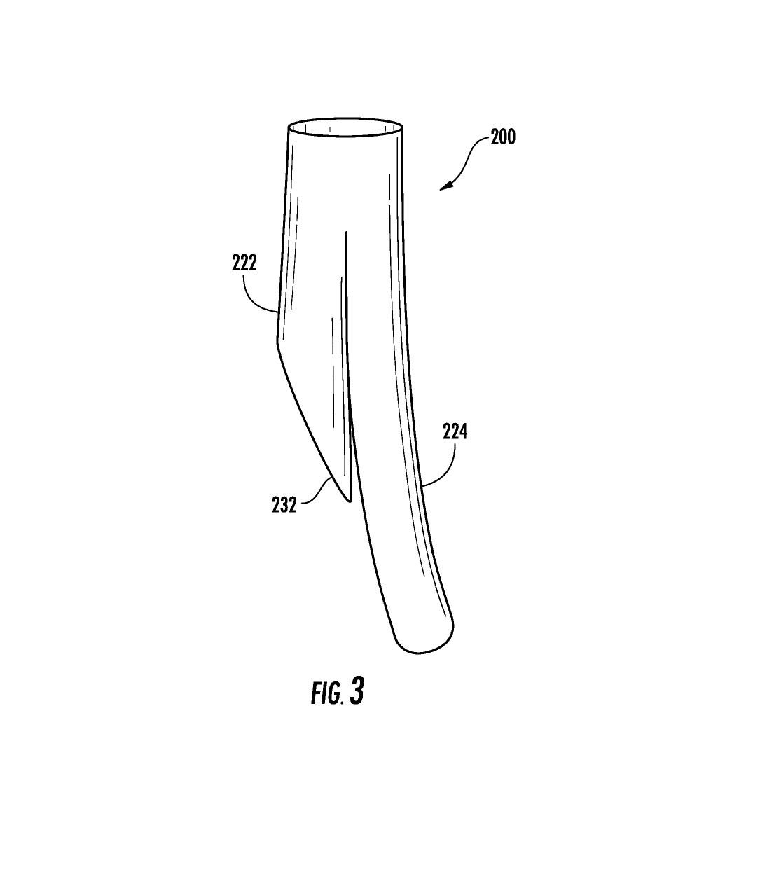

Referring to FIG. 3, a third embodiment of the stent graft is shown generally at 200. The flange or flared end 232 on the third embodiment 200 is flared medially and functions to deflect the wire towards the contralateral gate 222. The medial flare allows a larger surface/target toward which the wire could be aimed that is towards the ipsalateral limb 224 as opposed to away from the ipsilateral limb 224 in the second embodiment 100. This structure allows improved wire direction for contralateral gate cannulation, yet still leaves adequate opening above the origin of the contralateral iliac artery. The size of the medial flare 232 can be reduced in order to allow full deployment in small aneurysms and tighter spaces. The flare 232 may also be made shallow to accommodate smaller aneurysm and tighter spaces.

Therefore, it can be seen that the present invention provides a unique solution to the problem of providing a method of decreasing time during a critical phase of an operation to insert an abdominal aortic stent graft, which prevents excess risk to the patient. Specifically, the stent graft of the present invention includes a unique flange formed around the contralateral gate that funnels the catheter wire into stent graft permitting the contralateral limb to be positioned accurately and deployed. Additionally, the surgeon can spend less time focusing and planning on setting up the optimal angle or rotation for anticipated contralateral gate position prior to main body deployment in anticipation of wire cannulation of the contralateral gate.

Although this technique is intended for treatment of “aneurysm” disease of the aorta, it is not limited to just aortic aneurysm disease. This technique may be properly adapted to treat illiac disease or other diseases of the aorta.

It would be appreciated by those skilled in the art that various changes and modifications can be made to the illustrated embodiments without departing from the spirit of the present invention. All such modifications and changes are intended to be within the scope of the present invention.

I claim:

1. A stent graft, comprising:
a body portion with a sidewall and an opening at the top; the top of the sidewall configured and arranged to engage a wall of an aorta above an aneurysmal site;
an ipsilateral limb descending form the body portion, the ipsilateral limb configured and arranged to align with one of an iliac arteries; and
a contralateral gate having a flared opening configured and arranged to align with the other of the iliac arteries.

2. The stent graft of claim 1, wherein the flare forms a complete funnel.

3. The stent graft of claim 1, wherein the flare forms a partial funnel.

4. The stent graft of claim 1, wherein the contralateral gate flares medially.

5. The stent graft of claim 1, wherein the contralateral gate flares anteriorly.

6. The stent graft of claim 1, wherein the contralateral gate flares posteriorly.

7. The stent graft of claim 1, wherein the contralateral gate flares laterally.

8. The stent graft of claim 1, wherein the ipsilateral limb descends into one of the iliac arteries.

9. The stent graft of claim 1, further comprising a contralateral limb configured and arranged to deploy inside the contralateral gate.

10. The stent graft of claim 9, wherein the contralateral limb descends into the other of the iliac arteries.

11. A method of deploying a stent graft at an aneurysmal site of an abdominal aortic artery of a patient, comprising the steps of
deploying a stent graft at an aneurysmal site of an abdominal aortic artery of a patient, the stent graft having a body portion with a sidewall and an opening at the top, an ipsilateral limb descending form the body portion, and a contralateral gate having a flared opening;
positioning the top of the sidewall against a wall of the abdominal aorta above the aneurysmal site;
positioning the ipsilateral limb into one of an iliac arteries, and
positioning the contralateral gate in alignment with the other of the iliac arteries.

12. The method of claim 11, further comprising the step of manipulating a wire through the flared opening of the contralateral gate.

13. The method of claim 11, further comprising the step of manipulating a contralateral limb inside the contralateral gate.

14. The method of claim 13, further comprising the step of deploying a contralateral limb inside the contralateral gate.
